(19) United States
(12) Patent Application Publication
(10) Pub. No.: US 2024/0013385 A1
(43) Pub. Date: Jan. 11, 2024

(54) MEDICAL SYSTEM, METHOD FOR PROCESSING MEDICAL IMAGE, AND MEDICAL IMAGE PROCESSING APPARATUS

(71) Applicant: TERUMO KABUSHIKI KAISHA, Tokyo (JP)

(72) Inventors: Takanori TOMINAGA, Hadano Kanagawa (JP); Yuki SAKAGUCHI, Fujisawa Kanagawa (JP)

(21) Appl. No.: 18/471,211

(22) Filed: Sep. 20, 2023

Related U.S. Application Data
(63) Continuation of application No. PCT/JP2022/010152, filed on Mar. 9, 2022.

(30) Foreign Application Priority Data
Mar. 25, 2021 (JP) 2021-052012

(51) Int. Cl. G06T 7/00; A61B 5/00

(52) U.S. Cl. CPC G06T 7/0012; A61B 5/6852; A61B 5/489; A61B 5/742

(57) ABSTRACT

A medical system includes a catheter that includes a sensor and can be inserted into a luminal organ, a display apparatus, and an image processing apparatus configured to: generate an image of the luminal organ based on a signal output from the sensor of the catheter, input the generated image to a machine learning model and acquire an output indicating a type and a region of an object in the image, determine a size of a stent to be implanted into the luminal organ based on the type and region of the object, and cause the display apparatus to display information indicating the determined size of the stent.

### CROSS-REFERENCE TO RELATED APPLICATION

This application is a continuation of International Patent Application No. PCT/JP2022/010152 filed Mar. 9, 2022, which is based upon and claims the benefit of priority from Japanese Patent Application No. 2021-052012, filed on Mar. 25, 2021, the entire contents of which are incorporated herein by reference.

### FIELD

Embodiments described herein relate generally to a medical system, a method for processing a medical image of a luminal organ, and a medical image processing apparatus.

### BACKGROUND

An ultrasonic tomographic image of a blood vessel is generated by an intravascular ultrasound (IVUS) method using a catheter during an ultrasound examination of the blood vessel. Meanwhile, for the purpose of assisting diagnosis by a physician, a technology of adding information to a blood vessel image by image processing or machine learning has been developed. Such a technology includes a feature detection method for detecting a lumen wall, a stent, and the like included in the blood vessel image.

### SUMMARY OF THE INVENTION

Embodiments of the present disclosure provide a medical system or the like that provides information regarding a stent depending on an object included in a medical image that is obtained by scanning a luminal organ with a catheter.

According to an embodiment of the present invention, a medical system comprises a catheter that includes a sensor and can be inserted into a luminal organ; a display apparatus; and an image processing apparatus configured to: generate an image of the luminal organ based on a signal output from the sensor of the catheter, input the generated image to a machine learning model and acquire an output indicating a type and a region of an object in the image, determine a size of a stent to be implanted into the luminal organ based on the type and region of the object, and cause the display apparatus to display information indicating the determined size of the stent.

With this configuration, it is possible to provide a medical system or the like that provides information regarding a stent to be implanted into a luminal organ depending on an object included in a medical image that is obtained by scanning the luminal organ with a catheter.

### BRIEF DESCRIPTION OF THE DRAWINGS

FIG. 1 is a diagram illustrating a configuration example of an image diagnosis system.

FIG. 2 is a schematic diagram of an image diagnosis catheter.

FIG. 3 is an explanatory diagram illustrating a cross section of a blood vessel through which a sensor unit is inserted.

FIGS. 4A and 4B are explanatory diagrams of tomographic images.

FIG. 5 is a block diagram illustrating a configuration example of an image processing apparatus.

FIG. 6 is a diagram illustrating an example of a learning model.

FIG. 7 is a flowchart of information processing performed by an image processing apparatus.

FIG. 8 is a flowchart of a calculation procedure of a stent diameter.

FIG. 9 is a diagram illustrating a display example of information indicating an average lumen diameter.

FIG. 10 is a diagram exemplifying a selection screen for selecting a stent diameter calculation method.

FIG. 11 is a flowchart of a calculation procedure for calculating a stent length.

FIG. 12 is a diagram illustrating a display example of information indicating a landing zone.

FIG. 13 is a diagram illustrating a display example of information indicating a stent length.

FIG. 14 is a diagram illustrating a configuration example of an image diagnosis system according to a second embodiment.

FIG. 15 depicts a data structure of a stent inventory DB.

FIG. 16 is a flowchart of information processing performed by an image processing apparatus.

### DETAILED DESCRIPTION

Hereinafter, embodiments of the present disclosure will be described in detail with reference to the drawings. In each of the following embodiments, a cardiac catheter treatment as an endovascular treatment will be described as an example, but a luminal organ to be subjected to a catheter treatment is not limited to a blood vessel, and may be other luminal organs such as a bile duct, a pancreatic duct, a bronchus, and an intestine.

### First Embodiment

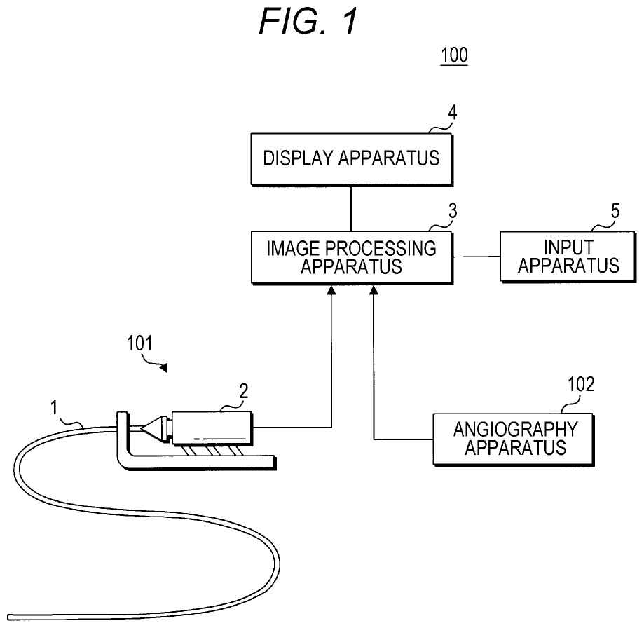

FIG. 1 is a diagram illustrating a configuration example of an image diagnosis system 100. In the present embodiment, the image diagnosis system 100 using a dual type catheter having functions of both intravascular ultrasound (IVUS) and optical coherence tomography (OCT) will be described. In the dual type catheter, a mode of acquiring an ultrasonic tomographic image only by IVUS, a mode of acquiring an optical coherence tomographic image only by OCT, and a mode of acquiring tomographic images by both IVUS and OCT are provided, and these modes can be switched and used. Hereinafter, an ultrasonic tomographic image and an optical coherence tomographic image are referred to as an IVUS image and an OCT image, respectively. In addition, an IVUS image and an OCT image are collectively referred to as tomographic images, and correspond to medical images.

The image diagnosis system 100 of the present embodiment includes an intravascular inspection apparatus 101, an angiography apparatus 102, an image processing apparatus 3, a display apparatus 4, and an input apparatus 5. The intravascular inspection apparatus 101 includes an image diagnosis catheter 1 and a motor drive unit (MDU) 2. The image diagnosis catheter 1 is connected to the image processing apparatus 3 via the MDU 2. The display apparatus 4 and the input apparatus 5 are connected to the image processing apparatus 3. The display apparatus 4 is, for example, a liquid crystal display (LCD), an organic EL (electro-luminescence) display, or the like, and the input apparatus 5 is, for example, a keyboard, a mouse, a trackball, a microphone, or the like. The display apparatus 4 and the input apparatus 5 may be integrated int a touch panel. Further, the input apparatus 5 and the image processing apparatus 3 may be integrated into one apparatus. Furthermore, the input apparatus 5 may be a sensor that receives a gesture input, a line-of-sight input, or the like.

The angiography apparatus 102 is connected to the image processing apparatus 3. The angiography apparatus 102 images a blood vessel from outside a living body of a patient using X-rays while injecting a contrast agent into the blood vessel of the patient to obtain an angiographic image that is a fluoroscopic image of the blood vessel. The angiography apparatus 102 includes an X-ray source and an X-ray sensor, and captures an X-ray fluoroscopic image of the patient by the X-ray sensor receiving X-rays emitted from the X-ray source. Note that the image diagnosis catheter 1 is provided with a radiopaque marker, and the position of the image diagnosis catheter 1 is visualized in the angiographic image using the marker. The angiography apparatus 102 outputs the angiographic image obtained by imaging to the image processing apparatus 3, and the angiographic image is displayed on the display apparatus 4 via the image processing apparatus 3. The display apparatus 4 displays the angiographic image and the tomographic image imaged using the image diagnosis catheter 1.

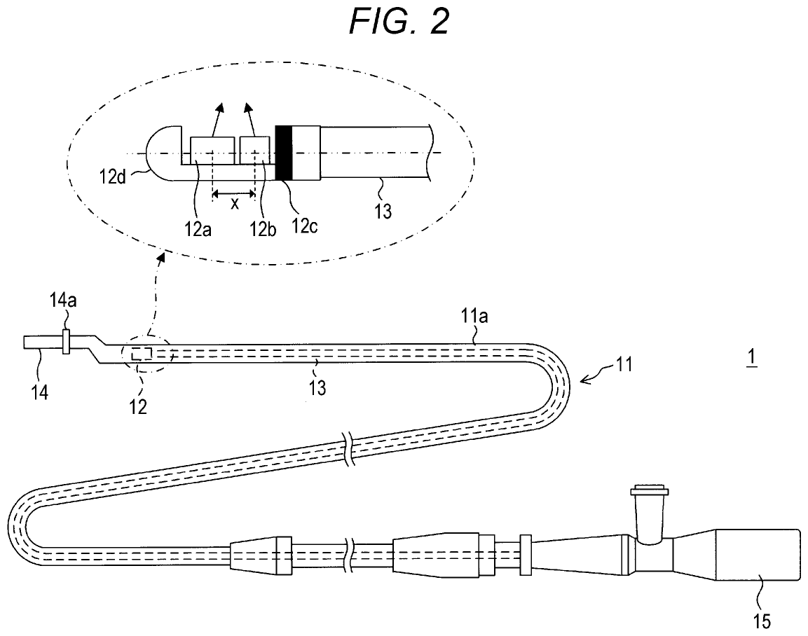

FIG. 2 is a schematic diagram of the image diagnosis catheter 1. Note that the region surrounded by a one-dot chain line on the upper side in FIG. 2 is an enlarged view of the region surrounded by a one-dot chain line on the lower side. The image diagnosis catheter 1 includes a probe 11 and a connector portion 15 disposed at an end of the probe 11. The probe 11 is connected to the MDU 2 via the connector portion 15. In the following description, a side far from the connector portion 15 of the image diagnosis catheter 1 will be referred to as a distal end side, and a side of the connector portion 15 will be referred to as a proximal end side. The probe 11 includes a catheter sheath 11a, and a guide wire insertion portion 14 through which a guide wire can be inserted is provided at a distal portion thereof. The guide wire insertion portion 14 constitutes a guide wire lumen, receives a guide wire previously inserted into a blood vessel, and guides the probe 11 to an affected part by the guide wire. The catheter sheath 11a forms a tube portion continuous from a connection portion with the guide wire insertion portion 14 to a connection portion with the connector portion 15. A shaft 13 is inserted into the catheter sheath 11a, and a sensor unit 12 is connected to a distal end side of the shaft 13.

The sensor unit 12 includes a housing 12d, and a distal end side of the housing 12d is formed in a hemispherical shape in order to suppress friction and catching with an inner surface of the catheter sheath 11a. In the housing 12d, an ultrasound transmitter and receiver 12a (hereinafter referred to as an IVUS sensor 12a) that transmits ultrasonic waves into a blood vessel and receives reflected waves from the blood vessel and an optical transmitter and receiver 12b (hereinafter referred to as an OCT sensor 12b) that transmits near-infrared light into the blood vessel and receives reflected light from the inside of the blood vessel are disposed. In the example illustrated in FIG. 2, the IVUS sensor 12a is provided on the distal end side of the probe 11, the OCT sensor 12b is provided on the proximal end side thereof, and the IVUS sensor 12a and the OCT sensor 12b are arranged apart from each other by a distance X along the axial direction on the central axis of the shaft 13 between two chain lines in FIG. 2. In the image diagnosis catheter 1, the IVUS sensor 12a and the OCT sensor 12b are attached such that a radial direction of the shaft 13 that is approximately 90 degrees with respect to the axial direction of the shaft 13 is set as a transmission/reception direction of an ultrasonic wave or near-infrared light. Note that the IVUS sensor 12a and the OCT sensor 12b are desirably attached slightly shifted from the radial direction so as not to receive a reflected wave or reflected light on the inner surface of the catheter sheath 11a. In the present embodiment, for example, as indicated by the arrows on the upper side of FIG. 2, the IVUS sensor 12a is attached with a direction inclined to the proximal end side with respect to a radial direction as an irradiation direction of the ultrasonic wave, and the OCT sensor 12b is attached with a direction inclined to the distal end side with respect to the radial direction as an irradiation direction of the near-infrared light.

An electric signal cable (not illustrated) connected to the IVUS sensor 12a and an optical fiber cable (not illustrated) connected to the OCT sensor 12b are inserted into the shaft 13. The probe 11 is inserted into the blood vessel from the distal end side. The sensor unit 12 and the shaft 13 can move forward or rearward inside the catheter sheath 11a and can rotate in a circumferential direction. The sensor unit 12 and the shaft 13 rotate about the central axis of the shaft 13 as a rotation axis. In the image diagnosis system 100, by using an imaging core including the sensor unit 12 and the shaft 13, a state of the blood vessel is observed by an ultrasonic tomographic image captured from the inside of the blood vessel or an optical coherence tomographic image captured from the inside of the blood vessel.

The MDU 2 is a drive device to which the probe 11 of the image diagnosis catheter 1 is detachably attached by the connector portion 15, and controls the operation of the image diagnosis catheter 1 inserted into the blood vessel by driving a built-in motor according to an operation by a medical worker. For example, the MDU 2 performs a pull-back operation of rotating the sensor unit 12 and the shaft 13 inserted into the probe 11 in the circumferential direction while pulling the sensor unit 12 and the shaft 13 toward the MDU 2 side at a constant speed. The sensor unit 12 continuously scans the inside of the blood vessel at predetermined time intervals while moving from the distal end side to the proximal end side by the pull-back operation and continuously captures a plurality of transverse tomographic images substantially perpendicular to the probe 11 at predetermined intervals. The MDU 2 outputs reflected wave data of an ultrasonic wave received by the IVUS sensor 12a and reflected light data received by the OCT sensor 12b to the image processing apparatus 3.

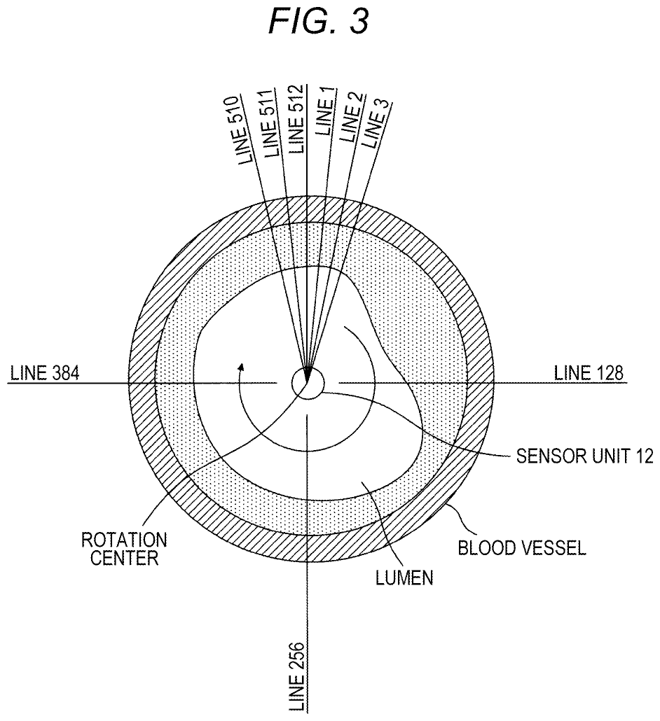

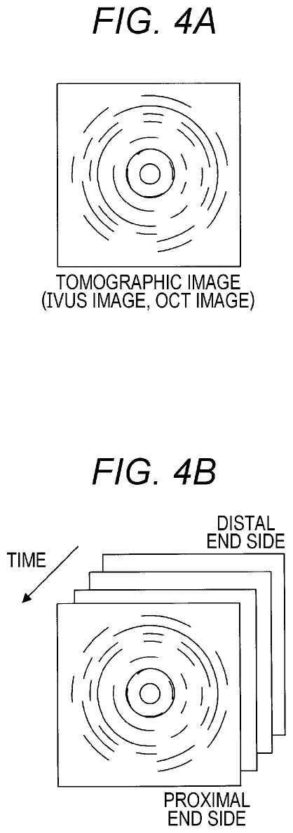

The image processing apparatus 3 acquires a signal data set which is the reflected wave data of the ultrasonic wave received by the IVUS sensor 12a and a signal data set which is reflected light data received by the OCT sensor 12b via the MDU 2. The image processing apparatus 3 generates ultrasound line data from the ultrasound signal data set, and generates an ultrasonic tomographic image of a transverse section of the blood vessel based on the generated ultrasound line data. In addition, the image processing apparatus 3 generates optical line data from the signal data set of the reflected light, and generates an optical tomographic image of a transverse section of the blood vessel based on the generated optical line data. Here, the signal data set acquired by the IVUS sensor 12a and the OCT sensor 12b and the tomographic image generated from the signal data set will be described. FIG. 3 is an explanatory diagram illustrating a cross section of the blood vessel through which the sensor unit 12 is inserted, and FIGS. 4A and 4B are explanatory diagrams of the tomographic images.

First, with reference to FIG. 3, operations of the IVUS sensor 12a and the OCT sensor 12b in the blood vessel, and signal data sets (i.e., ultrasonic line data and optical line data) acquired by the IVUS sensor 12a and the OCT sensor 12b will be described. When the imaging of the tomographic image is started in a state where the imaging core is inserted into the blood vessel, the imaging core rotates about the central axis of the shaft 13 as indicated by the arrow in FIG. 3. At this time, the IVUS sensor 12a transmits and receives an ultrasonic wave at each rotation angle. Lines 1, 2, . . . 512 indicate transmission/reception directions of ultrasonic waves at each rotation angle. In the present embodiment, the IVUS sensor 12a intermittently transmits and receives ultrasonic waves 512 times while rotating 360 degrees (i.e., one rotation) in the blood vessel. Since the IVUS sensor 12a acquires data of one line in the transmission/reception direction by transmitting and receiving an ultrasonic wave once, it is possible to obtain 512 pieces of ultrasonic line data radially extending from the rotation center during one rotation. The 512 pieces of ultrasonic line data are dense in the vicinity of the rotation center, but become sparse with distance from the rotation center. Therefore, the image processing apparatus 3 can generate a two-dimensional ultrasonic tomographic image as illustrated in FIG. 4A by generating pixels in an empty space of each line by known interpolation processing.

Similarly, the OCT sensor 12b also transmits and receives the measurement light at each rotation angle. Since the OCT sensor 12b also transmits and receives the measurement light 512 times while rotating 360 degrees in the blood vessel, it is possible to obtain 512 pieces of optical line data radially extending from the rotation center during one rotation. Moreover, for the optical line data, the image processing apparatus 3 can generate a two-dimensional optical coherence tomographic image similar to the IVUS image illustrated in FIG. 4A by generating pixels in a vacant space of each line by known interpolation processing. That is, the image processing apparatus 3 generates optical line data based on interference light generated by causing reflected light and, for example, reference light obtained by separating light from a light source in the image processing apparatus 3 to interfere with each other, and generates an optical tomographic image of the transverse section of the blood vessel based on the generated optical line data.

The two-dimensional tomographic image generated from the 512 pieces of line data in this manner is referred to as an IVUS image or an OCT image of one frame. Since the sensor unit 12 scans while moving in the blood vessel, an IVUS image or an OCT image of one frame is acquired at each position rotated once within a movement range. That is, since the IVUS image or the OCT image of one frame is acquired at each position from the distal end side to the proximal end side of the probe 11 in the movement range, as illustrated in FIG. 4B, the IVUS image or the OCT image of a plurality of frames is acquired within the movement range.

The image diagnosis catheter 1 has a radiopaque marker in order to confirm a positional relationship between the IVUS image obtained by the IVUS sensor 12a or the OCT image obtained by the OCT sensor 12b and the angiographic image obtained by the angiography apparatus 102. In the example illustrated in FIG. 2, a marker 14a is provided at the distal portion of the catheter sheath 11a, for example, the guide wire insertion portion 14, and a marker 12c is provided on the shaft 13 side of the sensor unit 12. When the image diagnosis catheter 1 configured as described above is imaged with X-rays, an angiographic image in which the markers 14a and 12c are visualized is obtained. The positions at which the markers 14a and 12c are provided are an example, the marker 12c may be provided on the shaft 13 instead of the sensor unit 12, and the marker 14a may be provided at a portion other than the distal portion of the catheter sheath 11a.

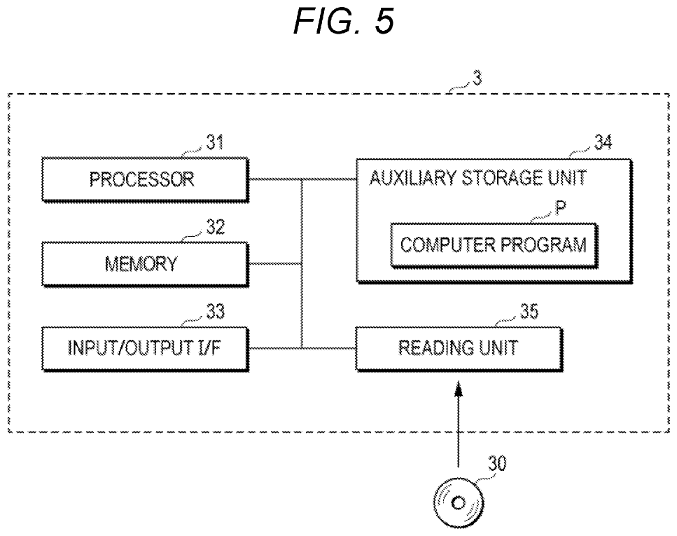

FIG. 5 is a block diagram illustrating a configuration example of the image processing apparatus 3. The image processing apparatus 3 includes a processor 31, a memory 32, an input/output interface (I/F) 33, an auxiliary storage unit 34, and a reading unit 35.

The processor 31 includes, for example, one or more central processing units (CPU), one or more micro-processing units (MPU), one or more graphics processing units (GPU), one or more general purpose graphics processing units (GPGPU), and one or more tensor processing units (TPU). The processor 31 is connected to each hardware component of the image processing apparatus 3 via a bus.

The memory 32 includes, for example, a static random access memory (SRAM), a dynamic random access memory (DRAM), or a flash memory, and temporarily stores data necessary for the processor 31 to execute arithmetic processing.

The input/output I/F 33 is an interface circuit to which the intravascular inspection apparatus 101, the angiography apparatus 102, the display apparatus 4, and the input apparatus 5 are connected. The processor 31 acquires the IVUS image and the OCT image from the intravascular inspection apparatus 101 via the input/output I/F 33, and acquires the angiographic image from the angiography apparatus 102. In addition, the processor 31 controls the input/output I/F 33 to output medical image signals of the IVUS image, the OCT image, or the angiographic image to the display apparatus 4, thereby displaying the medical image on the display apparatus 4. Furthermore, the processor 31 acquires information input to the input apparatus 5 via the input/output I/F 33.

For example, a communication unit including a wireless communication device supporting 4G, 5G, or Wi-Fi may be connected to the input/output I/F 33, and the image processing apparatus 3 may be communicably connected to an external server such as a cloud server connected to an external network such as the Internet via the communication unit. The image processing apparatus 3 may communicate with the external server via the communication unit and the external network, refer to medical data, paper information, and the like stored in a storage device included in the external server, and perform processing for providing support information. Alternatively, the processor 31 may cooperatively perform the processing in the present embodiment by performing, for example, inter-process communication with the external server.

The auxiliary storage unit 34 is a storage device such as a hard disk, an electrically erasable programmable ROM (EEPROM), or a flash memory. The auxiliary storage unit 34 stores a computer program P executed by the processor 31 and various data necessary for processing by the processor 31. Note that the auxiliary storage unit 34 may be an external storage device connected to the image processing apparatus 3. The computer program P may be stored in the auxiliary storage unit 34 at the manufacturing stage of the image processing apparatus 3, or the computer program distributed by a remote server device may be acquired by the image processing apparatus 3 through communication and stored in the auxiliary storage unit 34. The computer program P may be recorded in a non-transitory computer readable recording medium 30 such as a magnetic disk, an optical disk, or a semiconductor memory, and the reading unit 35 may read the computer program P from the recording medium 30 and store the computer program P in the auxiliary storage unit 34.

The image processing apparatus 3 may be composed of multiple processing devices. In addition, the image processing apparatus 3 may be a server client system, a cloud server, or a virtual machine operating as software. In the following description, it is assumed that the image processing apparatus 3 is one processing device. In the present embodiment, the image processing apparatus 3 is connected to the angiography apparatus 102 that images two-dimensional angiographic images. However, the present invention is not limited to that configuration, and the image processing apparatus 3 may be connected to any apparatus that images a luminal organ of a patient and the image diagnosis catheter 1 from a plurality of directions outside the living body.

In the image processing apparatus 3 of the present embodiment, the processor 31 reads and executes the computer program P stored in the auxiliary storage unit 34, thereby executing processing of generating the IVUS image based on the signal data set received from the IVUS sensor 12a and the OCT image based on the signal data set received from the OCT sensor 12b. Note that, since observation positions of the IVUS sensor 12a and the OCT sensor 12b are shifted at the same imaging timing as described later, the processor 31 executes processing of correcting the shift of the observation positions in the IVUS image and the OCT image. Therefore, the image processing apparatus 3 of the present embodiment provides an image that is easy to read by providing the IVUS image and the OCT image in which the observation positions are matched.

In the present embodiment, the image diagnosis catheter is a dual type catheter having functions of both intravascular ultrasound and optical coherence tomography, but is not limited thereto. The image diagnosis catheter may be a single type catheter having the function of either the intravascular ultrasound or the optical coherence tomography. Hereinafter, in the present embodiment, the image diagnosis catheter has the function of the intravascular ultrasound, and will be described based on the IVUS image generated by the IVUS function. However, in the description of the present embodiment, the medical image is not limited to the IVUS image, and the processing of the present embodiment may be performed using the OCT image as the medical image.

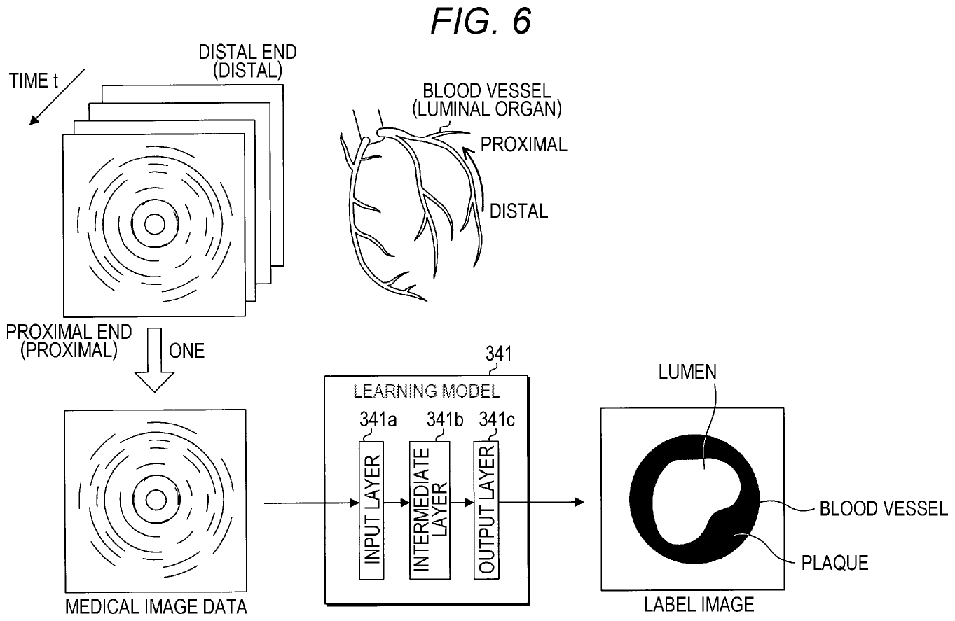

FIG. 6 is a diagram illustrating an example of a learning model 341. The learning model 341 is, for example, a segmentation neural network (NN) such as YOLO or R-CNN that performs object detection, semantic segmentation, or instance segmentation. Based on each IVUS image in the input IVUS image group, the learning model 341 outputs whether an object such as a stent or a plaque is included (i.e., present or absent) in the IVUS image, and in a case where the object is included (i.e., present), the learning model outputs a type or a class of the object, a region in the IVUS image, and estimation accuracy or a score.

The learning model 341 includes, for example, a trained convolutional neural network (CNN) by deep learning. The learning model 341 includes, for example, an input layer 341a to which a medical image such as an IVUS image is input, an intermediate layer 341b that extracts a feature amount of the image, and an output layer 341c that outputs information indicating a position and a type of an object included in the medical image. The input layer 341a of the learning model 341 has a plurality of neurons that receives an input of a pixel value of each pixel included in the medical image, and passes the input pixel value to the intermediate layer 341b. The intermediate layer 341b has a configuration in which a convolution layer for convoluting the pixel value of each pixel input to the input layer 341a and a pooling layer for mapping the pixel value convoluted by the convolution layer are alternately connected, and extracts the feature amount of an image while compressing pixel information of the medical image. The intermediate layer 341b passes the extracted feature amount to the output layer 341c. The output layer 341c includes one or a plurality of neurons that output the position, range, type, and the like of the image region of the object included in the image, for example, as a label image. The label image is, for example, an image in which a pixel corresponding to a plaque region is of a class “1” and pixels corresponding to other images are of a class “0”. Although the learning model 341 is the CNN, the configuration of the learning model 341 is not limited to the CNN. The learning model 341 may be, for example, a trained model having a configuration such as a neural network other than CNN, a fully convolution network (FCN) such as U-net, SegNet, SSD, SPPnet, a support vector machine (SVM), a Bayesian network, or a regression tree. Alternatively, the learning model 341 may input the image feature amount output from the intermediate layer to a support vector machine (SVM) to perform object recognition.

The learning model 341 can be generated by preparing training data in which a medical image including an object such as an epicardium, a side branch, a vein, a guide wire, a stent, a plaque deviating into a stent, a lipid plaque, a fibrous plaque, a calcified portion, blood vessel dissociation, thrombus, and haematoma and a label or label image indicating a position or region and a type of each object are associated with each other and causing an untrained neural network to perform machine learning using the training data. According to the learning model 341 configured in this manner, by inputting the medical image such as the IVUS image to the learning model 341, information indicating the position and type of the object included in the medical image can be obtained. In a case where no object is included in the medical image, the information indicating the position and the type is not output from the learning model 341. Therefore, by using the learning model 341, the processor 31 can acquire whether the object is included in the medical image input to the learning model 341, and in a case where the object is included, the control unit can acquire the type, the position or region in the medical image, and the estimation accuracy or score of the object. That is, by using the learning model 341 trained in this manner, it is possible to acquire the label image indicating the region of the plaque in units of pixels by inputting the IVUS image to the learning model 341 as illustrated in the present embodiment.

The processor 31 may input each IVUS image or the like to the learning model 341 one by one and perform processing, but may simultaneously input a plurality of consecutive frame images and simultaneously detect the region of the plaque from the plurality of frame images. For example, the processor 31 sets the learning model 341 as a 3D-CNN (for example, a 3D U-net) that handles three-dimensional input data. Then, the processor 31 processes the data as three-dimensional data in which the coordinates of the two-dimensional frame image are set to two axes and a time “t” at which each frame image is acquired is set to one axis. The processor 31 inputs a plurality of frame images (for example, 16 frames) for a predetermined unit time as one set to the learning model 341, and simultaneously outputs an image in which the region of the plaque is labeled with respect to each of the plurality of frame images. As a result, the region of the plaque can be detected in consideration of the frame images before and after being continuous in time series, and detection accuracy can be improved.

The processor 31 generates object information regarding the type and region of the object included in the IVUS image based on the information acquired from the learning model 341. Alternatively, the processor 31 may use the information output from the learning model 341 as the object information.

The processor 31 functions as a data input unit, a data processing unit, and a data output unit by executing the computer program P stored in the auxiliary storage unit 34. The data input unit acquires all the IVUS images generated in one pull-back operation. The data input unit may further acquire an angiographic image from the angiography apparatus 102. The IVUS image and the angiographic image acquired by the data input unit may be displayed on the display apparatus 4.

The data processing unit includes the learning model 341 having a segmentation function, and performs segmentation of the lumen and the blood vessel on each of the acquired IVUS images by using the learning model 341. The data processing unit specifies the region of the plaque based on the result of segmentation of the lumen and the blood vessel, and calculates a plaque burden and a stenosis rate. The data processing unit further calculates an average lumen diameter and a blood vessel diameter in the tomographic image of the blood vessel illustrated in each of the IVUS images by using the segmentation result. Based on the calculated plaque burden, stenosis rate, average lumen diameter, and blood vessel diameter, the data processing unit specifies, for example, a portion including a site where the plaque burden is the maximum value (here, the average lumen diameter is the minimum value) as a lesion, and specifies the reference portion in each of the regions on the distal side and the proximal side of the lesion. The data processing unit may specify, as the reference portion, a portion including a site where the plaque burden is the minimum value (here, the average lumen diameter is the maximum value) within a predetermined range where the separation distance from the lesion is, for example, 10 mm.

The data processing unit specifies the site (hereinafter referred to as the stent cover portion) to be covered by the stent based on the positions of the reference portions on the distal side and the proximal side in the axial direction of the blood vessel on which pull-back has been performed. The data processing unit can execute a plurality of calculation methods for calculating the stent diameter, such as Mean mid-wall reference, and calculates the diameter of the stent (hereinafter referred to as the stent diameter) based on the lumen diameters of the reference portions on the distal side and the proximal side using any of the calculation methods. The data processing unit calculates the length of the stent (hereinafter referred to as the stent length) based on the stent cover portion covered by the specified stent.

The data output unit outputs information such as various numerical values calculated by the data processing unit to the display apparatus 4, and these pieces of information are displayed on the display apparatus 4. The output information includes, for example, the IVUS image, the plaque burden, the average lumen diameter, the lesion, the reference portion, the stent cover portion, the stenosis rate, the stent diameter, and the stent length, and may further include calculated various values in calculating these pieces of information. Details of processing related to these functional units will be described in a flowchart and the like to be described later.

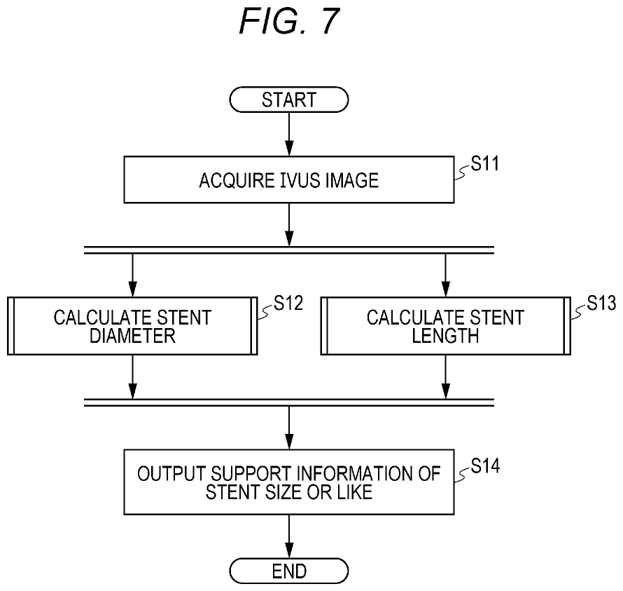

FIG. 7 is a flowchart of information processing performed by the processor 31. The processor 31 of the image processing apparatus 3 executes the following processing based on input data or the like output from the input apparatus 5 in response to an operation of an operator of the image diagnosis catheter 1 such as a physician.

The processor 31 acquires IVUS images (S11). The processor 31 reads an IVUS image group of a plurality of IVUS images corresponding to one pull-back operation.

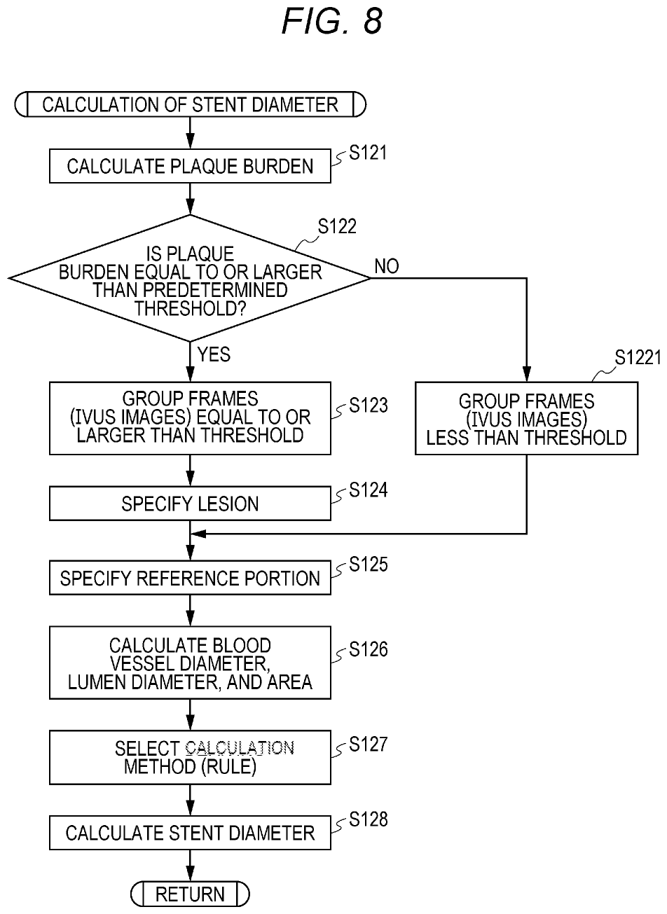

The processor 31 calculates a stent diameter (S12). FIG. 8 is a flowchart of a calculation procedure for calculating the stent diameter.

The processor 31 calculates a plaque burden (S121). For example, the processor 31 segments the lumen and the blood vessel from the acquired IVUS image using the learning model 341, and calculates the plaque burden. The plaque burden may be calculated by segmenting the lumen and the blood vessel to calculate these cross-sectional areas in the tomographic view, and dividing the area of the region other than the lumen by the area of the blood vessel.

The processor 31 determines whether the plaque burden is equal to or larger than a predetermined threshold (S122). The processor 31 determines whether the plaque burden is equal to or larger than a predetermined threshold, thereby classifying the plaque burden based on the threshold. The processor 31 classifies all the acquired IVUS images based on a predetermined threshold such as 40%, 50%, or 60% with respect to the calculated plaque burden.

When the plaque burden is equal to or larger than the predetermined threshold (YES in S122), the processor 31 groups frames (i.e., IVUS images) equal to or larger than the threshold (S123). The processor 31 groups the frames in which the plaque burden is equal to or larger than the threshold as the lesion. In a case where the lesion portions are scattered apart from each other, the lesion portions may be grouped (L1, L2, L3 . . . ). However, when the interval or distance between the groups is 0.1 to 3 mm or less, the groups may be the same.

The processor 31 specifies a group having the maximum value of plaque burden as the lesion (S124). The processor 31 specifies a group including a site where the maximum value of plaque burden, that is, the lumen diameter becomes the minimum value, as a lesion.

When the difference is not the predetermined threshold or more (NO in S122), that is, when the difference is less than the predetermined threshold, the processor 31 groups the frames (i.e., the IVUS images) less than the threshold (S1221). In a case where the value is less than the predetermined threshold, the processor 31 groups frames less than the threshold as a reference portion. In a case where the plaque burden to be the reference portion is scattered apart, the plaque burden may be grouped (R1, R2, R3, . . . ). However, when the interval between the groups is 0.1 to 3 mm or less, the groups may be the same.

The processor 31 specifies each of the groups on the distal side and the proximal side of the lesion as the reference portion (S125). For example, the processor 31 classifies whether the value is equal to or larger than the plaque burden threshold for all the IVUS images according to the determination result, and then specifies each of the groups on the distal side and the proximal side as a reference portion with respect to the lesion. The processor 31 specifies each group positioned on the distal side and the proximal side of the specified lesion among the plurality of grouped reference portions as a reference portion for comparing with the lesion.

The processor 31 calculates the blood vessel diameters (EEM), the lumen diameters, and the areas of the distal and proximal reference portions (S126). In this case, the length between the reference portions, that is, the length from the distal side reference portion to the proximal side reference portion may be set to be, for example, 10 mm at the maximum.

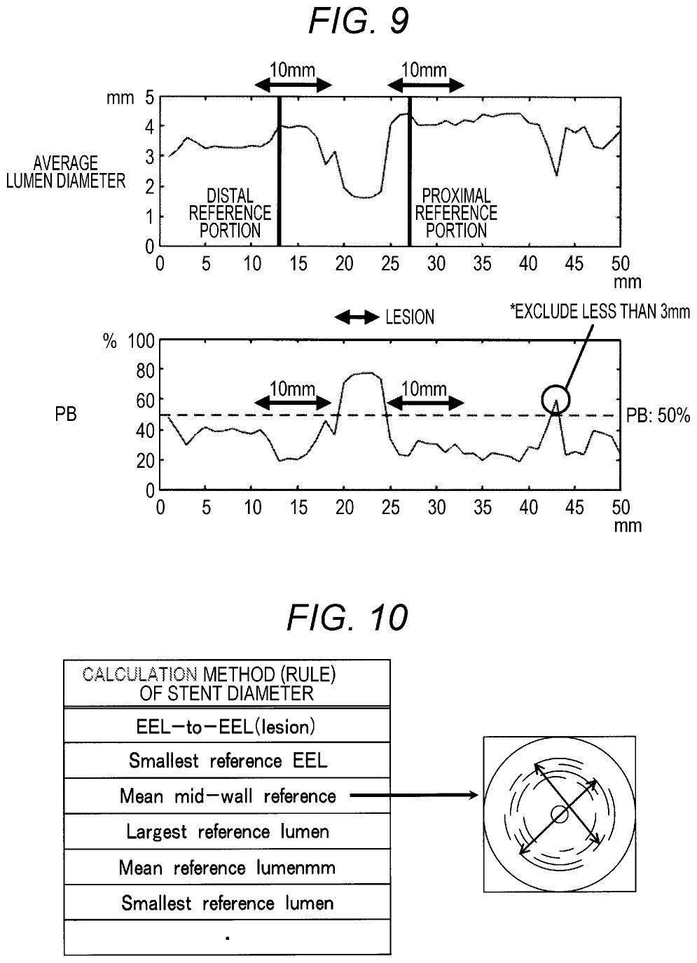

FIG. 9 is a diagram illustrating a display example of information indicating an average lumen diameter. In the display example, a graph of the average lumen diameter and a graph of plaque burden (PB) are displayed side by side vertically. The horizontal axis indicates the length of the blood vessel. When the plaque burden threshold is 50%, a site exceeding the threshold is specified as a lesion. With respect to the lesion, sites including respective points at which the average lumen diameter is maximized at sites within 10 mm on the distal side and the proximal side are specified as the distal reference portion and the proximal reference portion. By displaying such information, it is possible to assist the operator in specifying the reference portion. As illustrated in the present embodiment, the lesion may be a portion having the plaque burden of 50% or more, for example, and may be a group continuous for 3 mm or more, for example. The reference portion may be a portion including a site having the largest average lumen diameter within 10 mm front of and back of the lesion. When there is a large side branch in the blood vessel and the diameter of the blood vessel greatly changes, the reference portion may be specified between the lesion and the side branch. In specifying the reference portion, the image illustrated in the drawing may be displayed on the display apparatus 4, and correction by the operator may be accepted. In addition, when the image is displayed on the display apparatus 4, a portion having a large side branch may be presented.

The processor 31 may acquire information about a correction to the specified distal reference portion or proximal reference portion. For example, on the screen displaying the information such as the average lumen diameter described above, the processor 31 may acquire information about a correction input by the operator (e.g., physician) of the image diagnosis catheter or the like with respect to the position of the specified lesion, the distal reference portion, or the proximal reference portion in the axial direction of the blood vessel, or the blood vessel diameter or the lumen diameter at the site. With this configuration, it is possible for the operator to review and correct the information presented. When a correction is made to the reference portion, the processor 31 recalculates and redisplays information such as the average lumen diameter, and performs processing described later such as calculating the size of a stent to be placed.

The processor 31 selects any calculation method among a plurality of calculation methods for calculating the stent diameter (S127). A plurality of calculation methods for calculating the stent diameter are implemented as a submodule, a subroutine, a function library, or the like, to be executed by the processor 31.

FIG. 10 is an explanatory diagram illustrating a selection screen for selecting a stent diameter calculation method. The plurality of calculation methods include, for example, EEL-to-EEL (lesion), Smallest reference EEL, Mean mid-wall reference, Largest reference lumen, Mean reference lumen, and Smallest reference lumen, which are known calculation methods. The processor 31 may control the display apparatus 4 to output the names or types of the plurality of calculation methods in a menu format such as a list as illustrated in the present embodiment, for example, and the names may be displayed on the display apparatus 4 together with a function button or the like for receiving a selection operation.

The processor 31 calculates the diameter of the stent based on the specified lumen diameter and blood vessel diameter of the distal reference portion and the proximal reference portion using a selected calculation method such as Mean mid-wall reference (S128). In the present embodiment, the diameter of the stent is calculated using the selected calculation method, but the present invention is not limited thereto. The processor 31 may calculate the stent diameter by each of all the calculation methods prepared in advance based on the lumen diameter and the blood vessel diameter of the specified reference portion, and control the display apparatus 4 to display the stent diameter for each calculation method in a menu form such as a list. As a result, it is possible to present the stent diameter in each calculation method to a physician or the like and efficiently provide information related to determination support as to which calculation method to select.

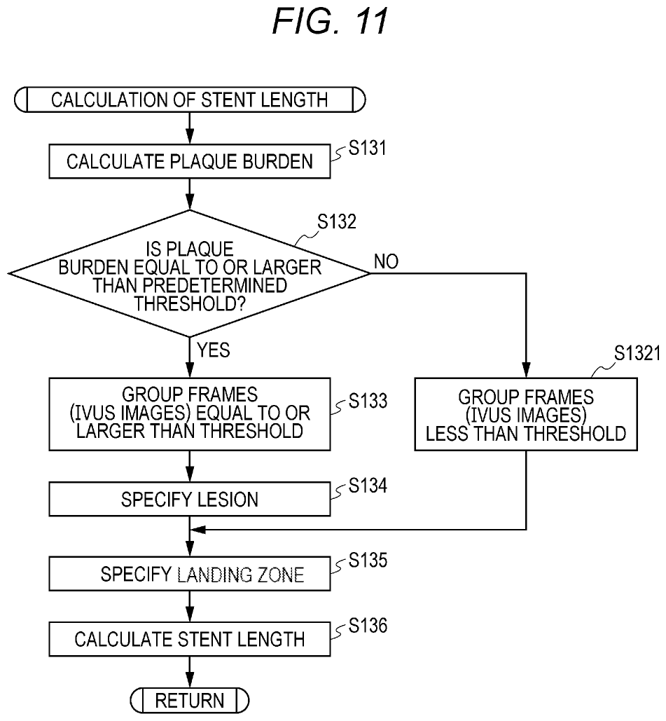

The processor 31 calculates a stent length (S13). FIG. 11 is a flowchart of a calculation procedure of the stent length.

The processor 31 calculates the plaque burden (S131). The processor 31 determines whether the plaque burden is equal to or larger than a predetermined threshold (S132). When the plaque burden is equal to or larger than the predetermined threshold (YES in S132), the processor 31 groups the frames equal to or larger than the threshold (S133). The processor 31 specifies a group having the maximum value of plaque burden as the lesion (S134). The processor 31 performs processing from S131 to S134 similarly to S121 to S124 described above. The processor 31 may set the processing results of S121 to S124 as the processing results of S131 to S134. That is, the processor 31 may perform common routines of the processing of S121 to S124 and the processing of S131 to S134.

When the plaque burden is not the predetermined threshold or more (NO in S132), that is, when the plaque burden is less than the predetermined threshold, the processor 31 groups the frames less than the threshold (S1321). When the plaque burden is less than the predetermined threshold, the processor 31 groups the frames less than the threshold as healthy portions. When the plaque burden to be healthy portions is scattered apart from each other, the plaque burden may be grouped (H1, H2, H3 . . . ). However, when the interval between the groups is 0.1 to 3 mm or less, the groups may be the same. The healthy portion and the reference portion specified in the processing of S1221 and S125 may be the same site.

The processor 31 specifies healthy portions (each H group) on the distal side and the proximal side of the specified lesion as a landing zone or implant zone in which the stent is to be implanted (S135). The processor 31 calculates the stent length capable of covering the landing zone (S136).

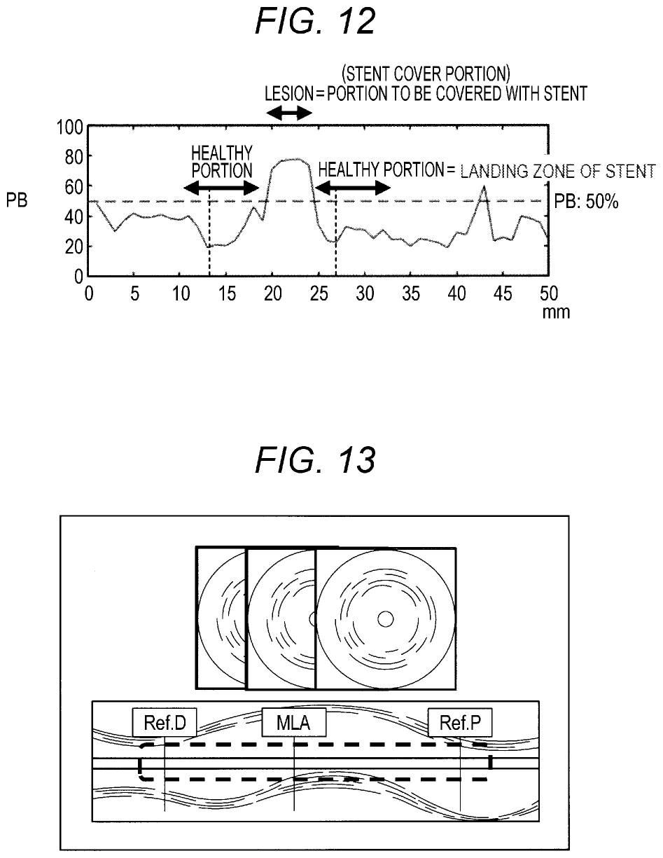

FIG. 12 is a diagram illustrating a display example of landing zone information. In the display example, a vertical axis represents the plaque burden, and a horizontal axis represents the length of the blood vessel subjected to pull-back. When the plaque burden threshold is 50%, a site exceeding the threshold is specified as a lesion. On the distal side and the proximal side with respect to the lesion, for example, portions where the average lumen diameter is the maximum at sites within 10 mm are specified as a distal healthy portion and a proximal healthy portion. The landing zone is specified based on the distal healthy portion and the proximal healthy portion, and the stent length is calculated based on the length of the specified landing zone.

The processor 31 may control the display apparatus 4 to display the illustrated image in the present embodiment and receive an input of information indicating a correction by the operator for the specified distal healthy portion or proximal healthy portion. When the correction is made to the healthy portion, the processor 31 recalculates the values displayed on the display apparatus 4 based on the correction. The processor 31 may accept a selection of one of a plurality of calculation methods prepared in advance similarly to the calculation of the stent diameter, and calculate the stent length using the selected calculation method.

The processor 31 outputs support information such as the stent size (S14). The processor 31 controls the display apparatus 4 to output the support information including the calculated stent size (i.e., the stent diameter and stent length). The support information may further include the IVUS image in each of longitudinal tomographic view and transverse tomographic view, the plaque burden, the average lumen diameter, the lesion, the reference portion, the stent cover portion, and the stenosis rate in addition to the stent size.

FIG. 13 is a diagram illustrating a display example of information indicating the stent length. The explanatory diagram is a screen display example when the support information is displayed on the display apparatus 4. In the display example, a transverse tomographic view which is a tomographic view in the axial direction of the blood vessel and a longitudinal tomographic view which is a tomographic view in the radial direction of the blood vessel are displayed side by side vertically. That is, the support information regarding stent implant includes a plurality of longitudinal tomographic views (i.e., cross-sectional areas in the radial direction of the blood vessel) of IVUS images and a transverse tomographic view (i.e., a cross-sectional area in the axial direction of the blood vessel) connecting these longitudinal tomographic views. In the transverse tomographic view, the distal reference portion “Ref. D” and the proximal reference portion “Ref. P” are illustrated, and a minimum lumen area (MLA) located between these reference portions is illustrated. By displaying such information, it is possible to assist the operator in performing stent implant.

Regarding the calculated stent length, as illustrated in the present embodiment, the processor 31 may control the display apparatus 4 to superimpose the stent cover portion indicated by dotted lines to be covered by the stent, for example, in the transverse tomographic view by the IVUS image. As a result, when the stent of the calculated length is inserted, a positional relationship between the lesion (MLA) covered by the stent and the distal and proximal reference portions (Ref D, Ref P) can be provided to a physician and the like. Various calculated values such as the stent size (i.e., the stent diameter and stent length), the plaque burden, the average lumen diameter, and the stenosis rate described above may be displayed in a superimposed manner or in an annotation manner on the IVUS image in the longitudinal tomographic view and transverse tomographic view, for example.

When the support information is displayed on the display apparatus 4 as illustrated in the present embodiment, the processor 31 may control the display apparatus 4 to display information regarding a plurality of candidates of stents. Furthermore, the processor 31 may control the display apparatus 4 to display a comment for selecting one of the plurality of candidates of stents. The processor 31 may control the display apparatus 4 to display information regarding the type of stent, the degree of expansion (e.g., low pressure/high pressure), and the like. When specifying the reference portion, the processor 31 may perform processing of specifying a suitable landing zone even in a case where there is no site where the plaque burden is, for example, less than 50%. In this case, the transverse section corresponding to the landing zone may be displayed. The processor 31 may perform processing of determining the position of the stent edge according to the plaque burden. The processor 31 may control the display apparatus 4 to display the expanded size of the selected stent. The processor 31 may simulate the placement of two stents, and in this case, the threshold of the plaque burden in a placement area of each stent may be changed and displayed.

According to the present embodiment, the image processing apparatus 3 specifies an adventitia and a lumen of a blood vessel as the type of an object using the learning model 341, and calculates an area ratio (i.e., a plaque burden) of the plaque based on the segmented regions of the adventitia and the lumen of the blood vessel. The image processing apparatus 3 generate information regarding the stent to be implanted into the blood vessel and applied to the plaque based on the plaque burden of the specified plaque. Since the information regarding the stent is, for example, the stent size including the diameter, length, and the like of the stent, it is possible to efficiently provide useful information to a physician and the like. In this manner, it is possible to generate the information such as the stent size based on the plaque specified by segmenting the IVUS image using the learning model 341, and it is possible to alleviate the difference depending on the subject or the operator of the image diagnosis catheter and provide information regarding an appropriate stent to a physician or the like.

According to the present embodiment, the image processing apparatus 3 specifies a lesion including a site where the plaque burden is maximized in all IVUS images corresponding to one pull-back, and specifies each of reference portions located on the distal side and the proximal side of the specified lesion. Therefore, in the axial direction of the blood vessel, the diameter and length of the stent corresponding to the range including the lesion where the plaque burden is the maximum value can be efficiently calculated. When the maximum value of the length of the reference portion used when calculating the diameter of the stent is restricted to, for example, 10 mm, the reference portion used when calculating the diameter of the stent and the reference portion used when calculating the length of the stent may be different sites. In this case, the healthy portion located on each of the distal side and the proximal side may be specified for the specified lesion separately from the reference portion for calculating the stent diameter, and the length of the stent may be calculated based on the specified healthy portion.

According to the present embodiment, the image processing apparatus 3 receives an input of a correction operation by the operator (e.g., physician or the like) of the image diagnosis catheter with respect to, for example, the position of the specified lesion, the distal reference portion, or the proximal reference portion in the axial direction of the blood vessel, or the blood vessel diameter (EEM) or the lumen diameter at the site. When receiving such correction, the image processing apparatus 3 calculates the diameter and length of the stent based on the corrected lesion, the distal reference portion, or the proximal reference portion. As described above, by calculating the diameter and length of the stent based on the lesion, the distal reference portion, and the proximal reference portion on the proximal side reflecting the correction by the physician and the like, it is possible to efficiently provide useful information to the physician and the like.

According to the present embodiment, the program executed by the processor 31 of the image processing apparatus 3 includes a module or a subroutine that performs a plurality of calculation methods for calculating the diameter of the stent based on the lumen diameter and the blood vessel diameter in either or both of the distal reference portion and the proximal reference portion. When calculating the diameter of the stent, the image processing apparatus 3 outputs the plurality of calculation methods to the display apparatus 4 in, for example, a list form, and displays the plurality of calculation methods on the display apparatus 4 together with a selection unit using a function button or the like for receiving selection of any of the calculation methods. As a result, the selection of the calculation method based on an operation by a physician or the like is received, and the stent diameter can be efficiently calculated using the selected calculation method.

### Second Embodiment

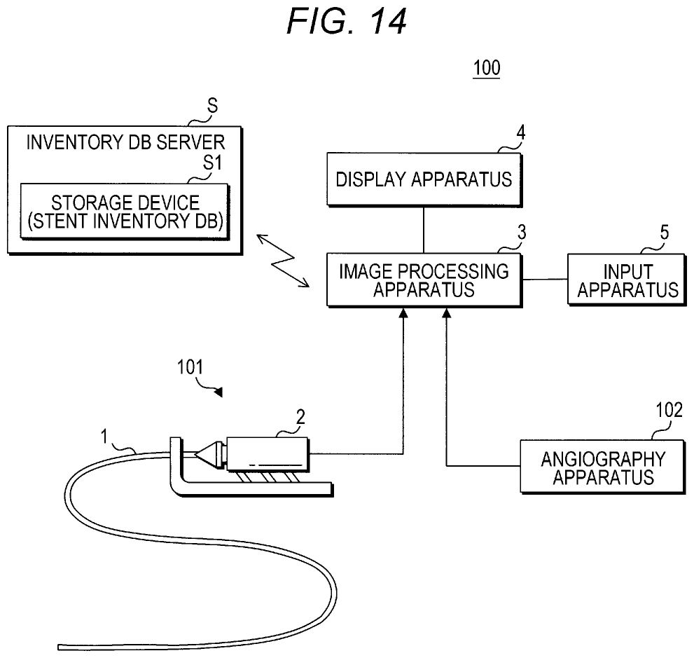

FIG. 14 is a diagram illustrating a configuration example of an image diagnosis system 100 according to a second embodiment. The image diagnosis system 100 is connected to an inventory database (DB) server S and communicates therewith in a wireless or wired manner. In the image diagnosis apparatus 100, for example, a communication unit including a wireless communication device supporting 4G, 5G, or Wi-Fi is connected to the input/output I/F 33 such as a USB port, and performs data communication with the inventory DB server S connected to the Internet and/or an intranet.

The inventory DB server S is an external server such as a cloud server, and includes a storage device S1. The storage device S1 of the inventory DB server S stores a stent inventory DB that stores data regarding the type and inventory of the stent. The image diagnosis system 100 can search and acquire data stored in the stent inventory DB by communicating with the inventory DB server S. In the present embodiment, the stent inventory DB that stores data regarding the stent type and the stent inventory is stored in the storage device S1 of the inventory DB server S. However, the present invention is not limited thereto, and the stent inventory DB may be stored in the auxiliary storage unit 34 of the image diagnosis system 100.

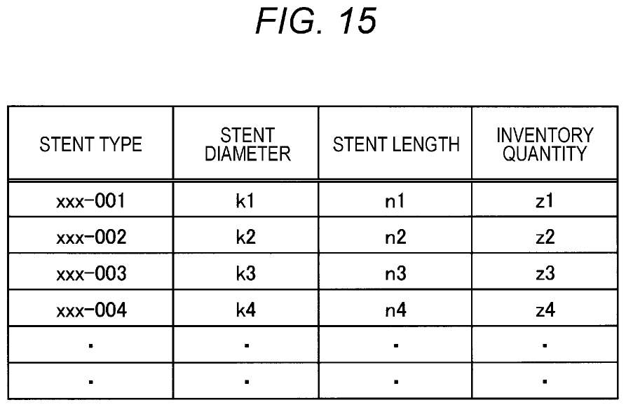

FIG. 15 is a diagram illustrating an example of the stent inventory DB. The stent inventory DB includes, for example, a stent type, a stent diameter, a stent length, and an inventory quantity as management items.

In a management item of the stent type, a type name of an existing commercial type is stored. In a management item of the stent diameter, the diameter of the stent having the type name stored in the same record is stored. In a management item of the stent length, the length of the stent having the type name stored in the same record is stored. In a management item of the inventory quantity, a current inventory quantity of the stent having the type name stored in the same record is stored. By accessing the inventory DB server S, the image diagnosis system 100 can search for the type name of the stent suitable for a predetermined stent size (i.e., a stent length and stent diameter) and determine the presence or absence of the inventory of the stent of the type name.

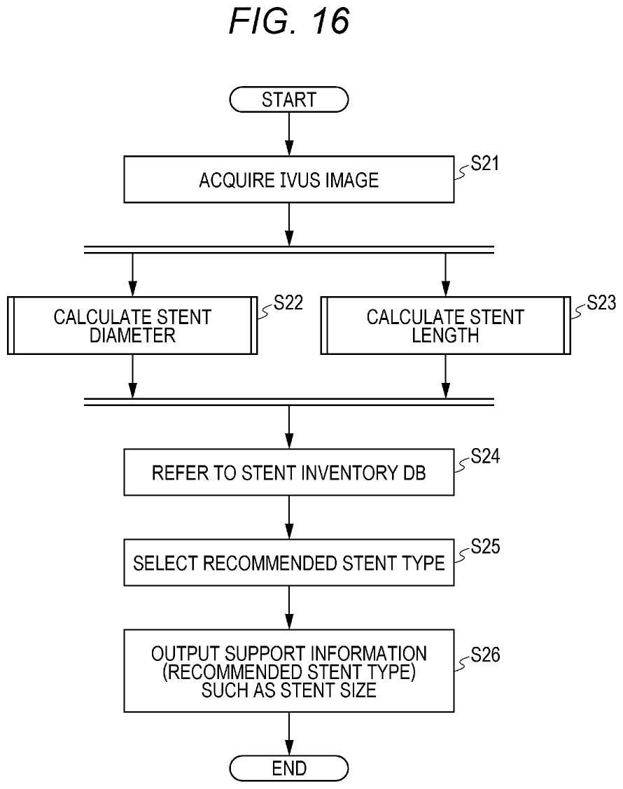

FIG. 16 is a flowchart of information processing performed by the processor 31. The processor 31 of the image processing apparatus 3 executes the following processing based on input data or the like output from the input apparatus 5 in response to an operation of an operator of the image diagnosis catheter 1 such as a physician.

The processor 31 acquires IVUS images (S21). The processor 31 calculates a diameter of a stent to be implanted based on the IVUS images (S22). The processor 31 calculates the stent length (S23). The processor 31 performs processing of S21 to S23 similarly to the processing of S11 to S13 of the first embodiment.

The processor 31 refers to the stent inventory DB (S24). The processor 31 selects a recommended stent type (S25). By accessing the inventory DB server S, the processor 31 searches the stent inventory DB based on the stent size (i.e., the stent diameter and stent length) calculated in the preprocessing, and selects a stent type currently in stock, which is as a stent type suitable for the stent size, as a recommended stent type. When selecting the recommended stent type, the processor 31 is not limited to the case of selecting a single recommended stent type, and may select a plurality of recommended stent types.

The processor 31 controls the display apparatus 4 to output the support information indicating the recommended stent type such as the stent size (S26). When outputting the support information, the processor 31 may control the display apparatus 4 to output and display the support information including information regarding the selected recommended stent type as in the first embodiment.

According to the present embodiment, the image processing apparatus 3 accesses, for example, the inventory DB server S that manages the inventory amount for each type of stent, and acquires the inventory amount of each type of stent. The image processing apparatus 3 selects, as the stent having a recommended size, the type of the stent that is suitable for the calculated diameter and length of the stent and has stock at present, for example. As a result, it is possible to efficiently provide information regarding the stent of an appropriate type to a physician or the like based on the calculated diameter and length (stent size) of the stent.

It should be understood that the embodiments disclosed herein are illustrative in all respects and are not restrictive. The technical features described in the examples can be combined with each other, and the scope of the present invention is intended to include all modifications within the scope of the claims and the scope equivalent to the claims.

What is claimed is:

1. A medical system comprising:
a catheter that includes a sensor and can be inserted into a luminal organ;
a display apparatus; and
an image processing apparatus configured to:
generate an image of the luminal organ based on a signal output from the sensor of the catheter,
input the generated image to a machine learning model and acquire an output indicating a type and a region of an object in the image,
determine a size of a stent to be implanted into the luminal organ based on the type and region of the object, and
cause the display apparatus to display information indicating the determined size of the stent.

2. The medical system according to claim 1, wherein the luminal organ is a blood vessel, and the object is a plaque between an adventitia and a lumen of the blood vessel.

3. The medical system according to claim 2, wherein the image processing apparatus is configured to:
calculate a plurality of plaque burdens of the plaque along the blood vessel,
specify a lesion in the plaque having a maximum plaque burden,
specify reference portions of the blood vessel located on a distal side and a proximal side with respect to the lesion,
determine diameters of the specified reference portions, and
determine a diameter of the stent to be displayed based on the determined diameters of the reference portions.

4. The medical system according to claim 3, wherein the image processing apparatus is configured to:
determine a landing zone of the stent based on locations of the reference portions, and
determine a length of the stent corresponding to the determined landing zone.

5. The medical system according to claim 3, wherein the image processing apparatus is configured to:
receive an input for correcting the diameters of the reference portions, and
determine the diameter of the stent based on the corrected diameters of the reference portions.

6. The medical system according to claim 3, wherein the image processing apparatus is configured to:
receive a selection of one of methods for calculating the diameter of the stent, and
calculate the diameter of the stent using the selected method.

7. The medical system according to claim 3, wherein the image processing apparatus is configured to:
store information indicating a plurality of types of stent each associated with a diameter thereof,
select one of the types of stent corresponding to the determined diameter of the stent, and
cause the display apparatus to display information indicating the selected one of the types of stent.

8. The medical system according to claim 7, wherein the image processing apparatus is configured to:
store information indicating an availability of each of the types of stent, and
select said one of the types of stent based on the availability.

9. The medical system according to claim 2, wherein the image processing apparatus is configured to:
calculate a plurality of plaque burdens of the plaque along the blood vessel,
specify a lesion in the plaque having a plaque burden that is greater than or equal to a threshold value,
specify healthy portions of the blood vessel located on a distal side and a proximal side with respect to the lesion and having a plaque burden less than the threshold value,
determine a landing zone of the stent based on locations of the healthy portions, and
determine a length of the stent corresponding to the determined landing zone.

10. The medical system according to claim 9, wherein the image processing apparatus is configured to:
receive an input for correcting the locations of the healthy portions, and
determine the length of the stent based on the corrected locations of the healthy portions.

11. A method for processing a medical image of a luminal organ, comprising:
generating an image of the luminal organ based on a signal output from a sensor of a catheter inserted into the luminal organ;
inputting the generated image to a machine learning model and acquiring an output indicating a type and a region of an object in the image;
determining a size of a stent to be implanted into the luminal organ based on the type and region of the object; and
displaying information indicating the determined size of the stent.

12. The method according to claim 11, wherein the luminal organ is a blood vessel, and the object is a plaque between an adventitia and a lumen of the blood vessel.

13. The method according to claim 12, further comprising:
calculating a plurality of plaque burdens of the plaque along the blood vessel;
specifying a lesion in the plaque having a maximum plaque burden;
specifying reference portions of the blood vessel located on a distal side and a proximal side with respect to the lesion;
determining diameters of the specified reference portions; and
determining a diameter of the stent to be displayed based on the determined diameters of the reference portions.

14. The method according to claim 13, further comprising:
determining a landing zone of the stent based on locations of the reference portions; and
determining a length of the stent corresponding to the determined landing zone.

15. The method according to claim 13, further comprising:
receiving an input for correcting the diameters of the reference portions; and
determining the diameter of the stent based on the corrected diameters of the reference portions.

16. The method according to claim 13, further comprising:
receiving a selection of one of methods for calculating the diameter of the stent, wherein
the diameter of the stent is determined using the selected method.

17. The method according to claim 13, further comprising:
storing information indicating a plurality of types of stent each associated with a diameter thereof;
selecting one of the types of stent corresponding to the determined diameter of the stent; and
displaying information indicating the selected one of the types of stent.

18. The method according to claim 17, further comprising:
storing information indicating an availability of each of the types of stent, wherein
said one of the types of stent is selected based on the availability.

19. The method according to claim 12, further comprising:
calculating a plurality of plaque burdens of the plaque along the blood vessel;
specifying a lesion in the plaque having a plaque burden that is greater than or equal to a threshold value;
specifying healthy portions of the blood vessel located on a distal side and a proximal side with respect to the lesion and having a plaque burden less than the threshold value;
determining a landing zone of the stent based on locations of the healthy portions; and
determining a length of the stent corresponding to the determined landing zone.

20. A medical image processing apparatus comprising:
an interface circuit connectable to a display apparatus and a catheter that includes a sensor and can be inserted into a luminal organ; and
a processor configured to:
generate an image of the luminal organ based on a signal output from the sensor of the catheter,
input the generated image to a machine learning model and acquire an output indicating a type and a region of an object in the image,
determine a size of a stent to be implanted into the luminal organ based on the type and region of the object, and
cause the display apparatus to display information indicating the determined size of the stent.
